(19) United States
(12) Patent Application Publication
(10) Pub. No.: US 2007/0162065 A1
(43) Pub. Date: Jul. 12, 2007

(54) Disposable lancet device

(75) Inventors: Fu-Yuan Li, Wantan Hsiang (TW); Fu-Chin Lee, Wantan Hsiang (TW)

Correspondence Address: PATENTTM.US, P. O. BOX 82788, PORTLAND, OR, 97282-0788, US

(73) Assignees: Mu-Shen Chen; Fu-Yuan Li; Fu-Chin Lee

(21) Appl. No.: 11/331,521

(22) Filed: Jan. 12, 2006

(51) Int. Cl. A61B 17/32

(52) U.S. Cl. 606/182

(57) ABSTRACT

The disposable lancet device has a casing, a lancet assembly, a spring and an actuator is provided. The casing is hollow and has a front end, an open rear end, two inner side surfaces and two converging inner linear surfaces. Each inner linear surface has a shoulder and an inclined limit. The lancet assembly is mounted slidably in the casing and has a longitudinal hub with two resilient tabs that abut the shoulders and a needle cap. The spring presses against the longitudinal hub. The actuator presses against the spring and has a pushbutton assembly and two actuating tabs that press against and release the resilient tabs from the shoulders. The needle is retracted by the resilient tabs pressing against the inclined limits. Therefore, the used needle will not protrude from the casing to prick a user cannot be used again.

### BACKGROUND OF THE INVENTION

1. Field of the Invention

The present invention relates to a lancet device, and especially to a lancet device for a single use, which cannot be used after the initial use and must be discarded.

2. Description of the Related Art

With modem developments in medical technology, rapid and conveniently operated measurement devices only need a very small amount of blood to measure, record and readout parameters of the blood. Therefore, a very small amount of blood, even less than a single drop, is obtained by pricking a person's skin. A conventional pencil-like lancet device is used to obtain small samples of blood. The lancet device comprises a casing, a disposable lancet, a drive assembly and an actuating button. The casing is tubular and has an open distal end and an open proximal end. The disposable lancet is mounted in and selectively protrudes from the open distal end of the casing to prick a person's skin so a small blood sample can be obtained and has a shaft and a needle. The shaft has a proximal end and a distal end. The needle protrudes from the distal end. The drive assembly is mounted in the casing, abuts the proximal end of the shaft of the disposable lancet and drives the needle through the open distal end of the casing to prick a person's skin. The actuating button is mounted in and protrudes from the casing, is attached to and actuates the drive assembly. However, the lancet device can be cocked and used to take another blood sample without changing the disposable lancet. If the lancet device is used to take a blood sample from another person without replacing the used disposable lancet, the contaminated lancet may infect people from whom a blood sample is obtained. Furthermore, a person replacing a used disposable lancet can easily be pricked by the used disposable lancet when replacing the disposable lancet.

### SUMMARY OF THE INVENTION

The objective of the present invention is to provide a disposable lancet device that can only be used once, thereby decreasing the danger of infection.

To achieve the foregoing objective, a disposable lancet device having a casing, a lancet assembly, a spring and an actuator is provided. The casing is hollow and has a front end, an open rear end, two inner side surfaces and two converging inner linear surfaces. Each inner linear surface has a shoulder and an inclined limit. The lancet assembly is mounted slidably in the casing and has a longitudinal hub with two resilient tabs that abut the shoulders and a needle cap. The spring presses against the longitudinal hub. The actuator presses against the spring and has a pushbutton assembly and two actuating tabs that press against and release the resilient tabs from the shoulders. The needle is retracted by the resilient tabs pressing against the inclined limits. Therefore, the used needle will not protrude from the casing to prick a user cannot be used again.

Other objectives, advantages and novel features of the invention will become more apparent from the following detailed description when taken in conjunction with the accompanying drawings.

### BRIEF DESCRIPTION OF THE DRAWINGS

FIG. 1 is a perspective view of a disposable lancet device in accordance with the present invention;

FIG. 2 is an exploded perspective view in partial section of the disposable lancet device in FIG. 1;

FIG. 3 is a side view in partial section of the disposable lancet device in FIG. 1;

FIG. 4 is an operational side view in partial section of the disposable lancet device in FIG. 1 with an actuator pressed;

FIG. 5 is an operational side view in partial section of the disposable lancet device in FIG. 1 with a needle protruding from a casing; and

FIG. 6 is an operational side view in partial section of the disposable lancet device in FIG. 1 with the needle retracted into the casing.

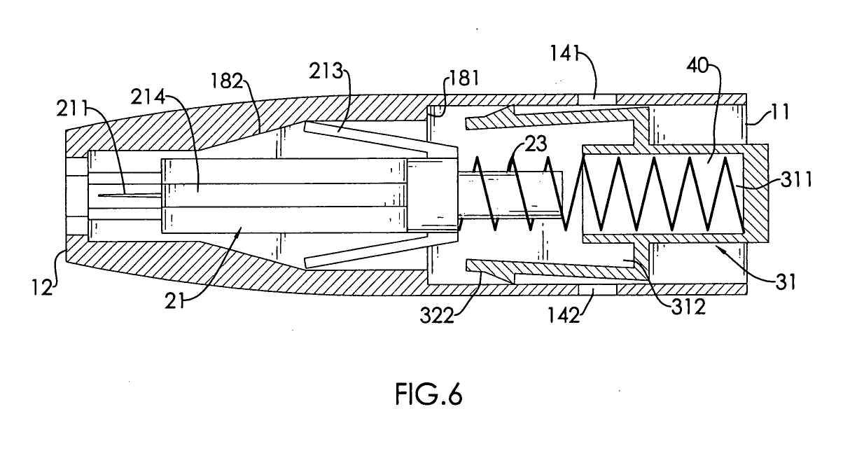

### DETAILED DESCRIPTION OF THE PREFERRED EMBODIMENTS

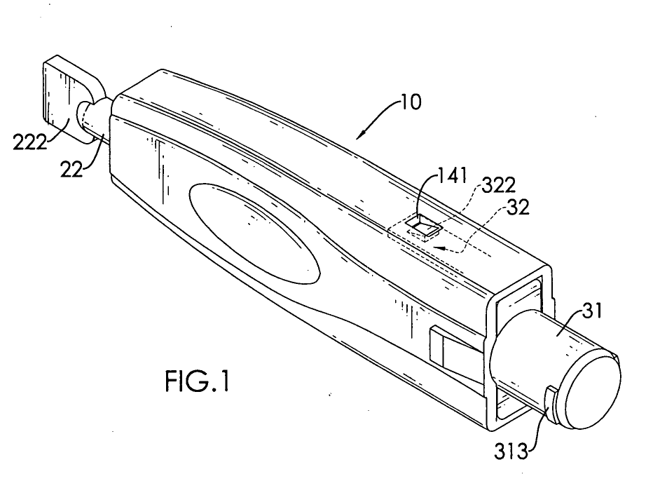

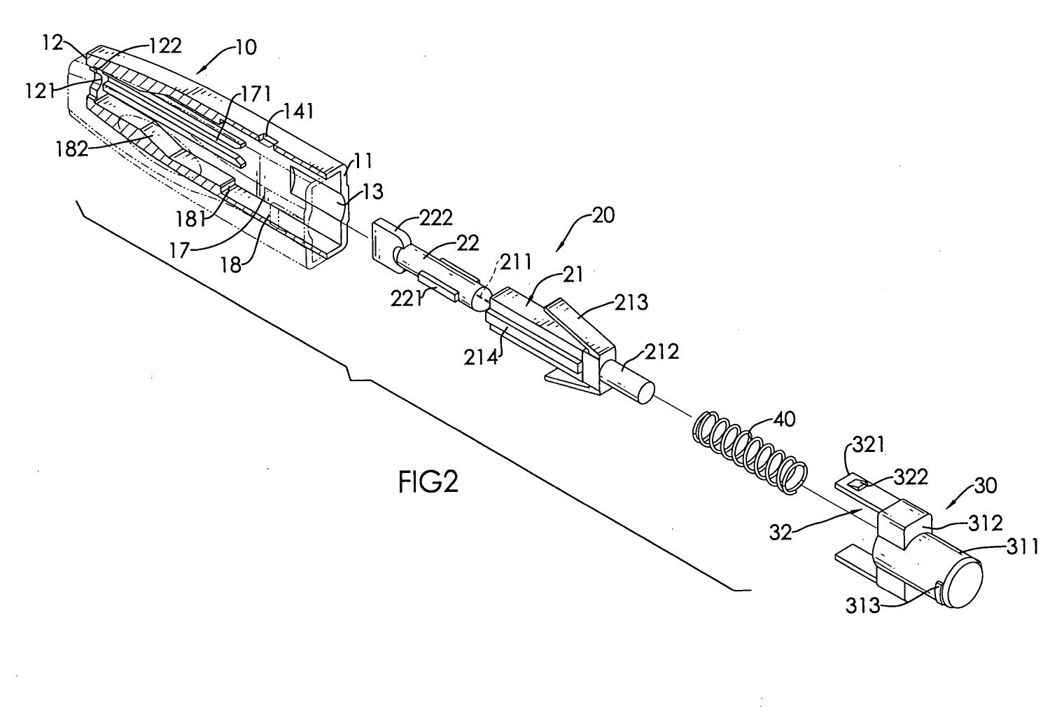

With reference to FIGS. 1 and 2, a disposable lancet device in accordance with the present invention comprises a casing (10), a lancet assembly (20), a spring (40) and an actuator (30).

The casing (10) is a hollow elongated hexahedron, may be slightly tapered and comprises a front end (12), an open rear end (11), a top, a bottom, two inner side surfaces (17), two inner linear surfaces (18) and two optional recesses (13).

The front end (12) has an opening (121) and two optional vertical notches (122). The opening (121) is circular. The vertical notches (122) communicate with and extend radially from the opening (121) opposite to each other.

The top has a through hole (141) formed toward the open rear end (11).

The bottom has a through hole (142) corresponding to the through hole (141) in the top.

The inner side surfaces (17) face each other and have two optional guide slots (171). The guide slots (171) are formed respectively on the inner side surfaces (117), face each other and communicate with the front end (12).

The inner linear surfaces (18) face each other, are between the inner side surfaces (17), extend from the open rear end (11) to the front end (12) and converge as they approach the front end (12), and each inner linear surface (18) has a rear longitudinal surface, a front longitudinal surface, a middle longitudinal surface, a shoulder (181) and an inclined limit (182). The front longitudinal surface connects to the front end (12) of the casing (10). The rear longitudinal surface connects to the open rear end (11) of the casing (10). The middle longitudinal surface is formed between the front and rear longitudinal surfaces. The shoulder (181) is formed between the rear longitudinal surface and the middle longitudinal surface. The inclined limit (182) is formed between the middle longitudinal surface and the front longitudinal surface.

The recesses (13) are formed in the casing (10), communicate with the open rear end (11) and may be formed respectively in the inner side surfaces (17) or respectively in the rear longitudinal surfaces of the inner linear surfaces (18).

The lancet assembly (20) is a shaft-like assembly, is mounted in the casing (10) through the open rear end (11) and comprises a longitudinal hub (21) and an optional needle cap (22).

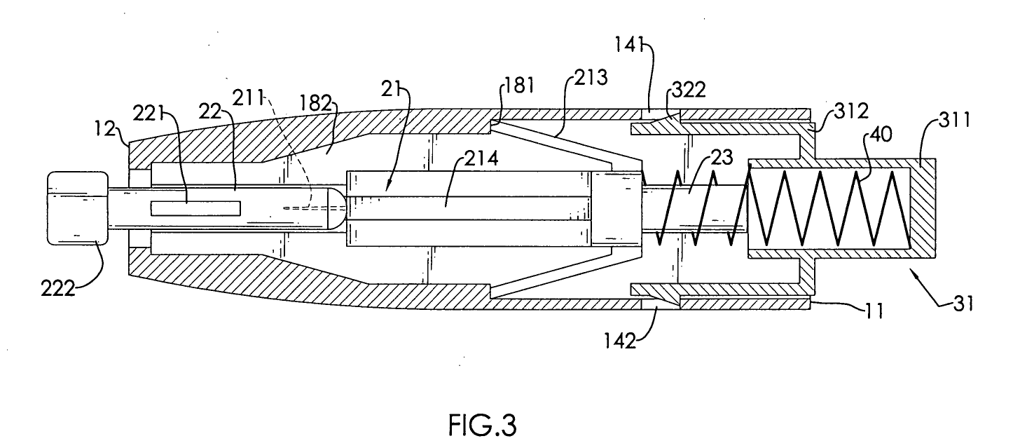

The longitudinal hub (21) has a top, a bottom, two sides, a front end, a rear end, a needle (211), a post (212), two resilient tabs (213) and two optional ribs (214). The needle (211) is mounted in and protrudes coaxially from the front end. The post (212) is formed on and extends coaxially from the rear end. The resilient tabs (213) are formed respectively on the top and bottom of the longitudinal hub (21) at the rear end, extent toward the front end and diverge from each other, and each resilient tab (213) has a front edge. With further reference to FIG. 3, the front edges of the resilient tabs (213) abut the shoulders (181) to hold the lancet assembly (20) in the casing (10) when the lancet assembly (20) is mounted in the open rear end (11) of the casing (10). The ribs (214) are longitudinally formed respectively on the sides of the longitudinal hub (21) and slidably engage the guide slots (171) on the inner side surfaces (17).

The needle cap (22) attaches to and extends from the front end of the longitudinal hub (21), covers the needle (211), is removed from the lancet assembly (20) through the front end (12) of the casing (10) before the disposable lancet device is used and comprises a cylindrical body, two longitudinal keys (221) and a tab (222). The cylindrical body is attached to the front end of the longitudinal hub (21), covers the needle (211) and has a front end, a rear end and a longitudinal outer surface. The rear end is attached to the front end of the longitudinal hub (21), covers the needle (211) and is detached from the longitudinal hub (21) by twisting the cylindrical body. The longitudinal keys (221) are formed on the longitudinal outer surface of the body of the needle cap (22) opposite to each other and align with and extend through the vertical notches (122) in the front end (12) of the casing (10). The tab (222) is formed on and extends from the front end of the longitudinal body of the needle cap (22) and is used to twist the longitudinal body and detach the needle cap (22) from the longitudinal hub (21).

The spring (40) is mounted around the post (212) of the longitudinal hub (21) of the lancet assembly (20), presses against the rear end of the longitudinal hub (21) and has a front end and a rear end.

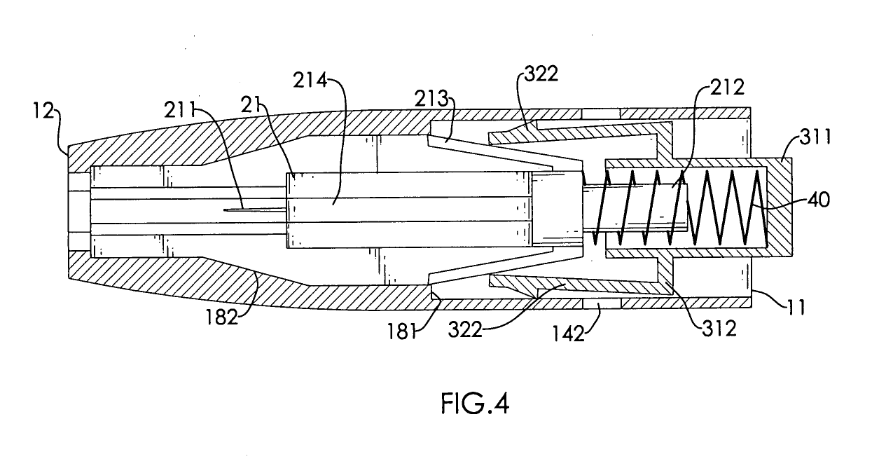

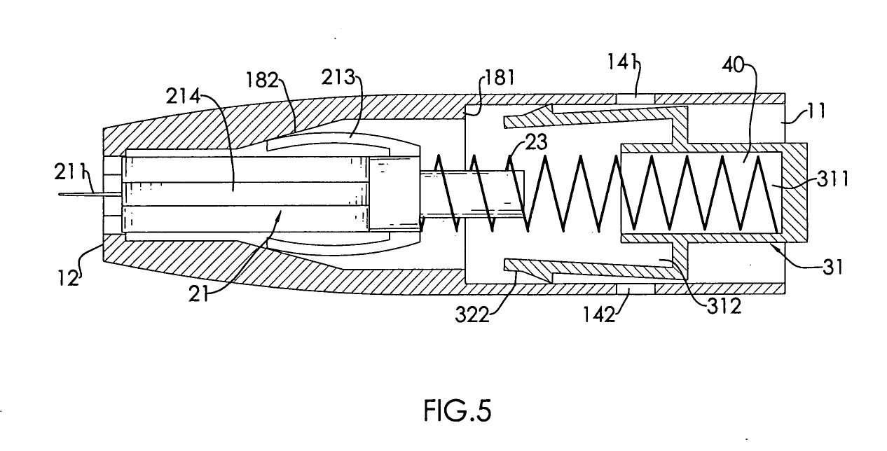

With further reference to FIG. 4 and 5, the actuator (30) is mounted slidably in the recesses (13) in the rear end of the casing (10) and around the rear end of the longitudinal hub (21), compresses the spring (40), squeezes the resilient tabs (213) toward each other to release the front edges of the resilient tabs (213) from the shoulders (181) on the inner linear surfaces (18) so the longitudinal hub (21) can be driven forward by the spring (40) and make the needle (211) protrude from the opening (121) to pierce a person's skin. After the needle (211) pierces a person's skin, the resilient tabs (213) press against the inclined limits (182) and draw the needle (211) back into the casing (10). The actuator (30) comprises a pushbutton assembly (31) and two actuating tabs (32).

The pushbutton assembly (31) is mounted slidably in the open rear end (11) and the recesses (13) in the casing (10) and has a cylindrical body (311), two mounting keys (312) and two optional circumferential stops (313).

The cylindrical body (311) is mounted slidably in the recesses (13) in the open rear end (11) of the casing (10), is hollow, holds the rear end of the spring (40), compresses the spring (40) when pressed into the casing (10) and has an open front end and a closed rear end. The open front end is mounted around the spring (40). The rear end of the cylindrical body (311) protrudes from the open rear end (11) of the casing (10).

The mounting keys (312) are formed at and protrude out from the front end of the cylindrical body (311), are slidably mounted between the inner side surfaces (17) of the casing (10), may be slidably mounted respectively in the recesses (13) in the rear longitudinal surfaces of the inner linear surfaces (18) and slidably press respectively against the rear longitudinal surfaces of the inner linear surfaces (18). Each mounting key (312) has a front outer edge.

The circumferential stops (313) are formed near and protrude out from the closed rear end of the cylindrical body (311) and keep the actuator (30) from being pressed completely into the casing (10) by abutting the open rear end (11) of the casing (10) when the actuator (30) is pressed into the casing (10).

The actuating tabs (32) are formed on and extend longitudinally respectively from the front outer edges of the mounting keys (312), are slidably mounted respectively against the rear longitudinal surfaces of the inner linear surfaces (18) of the casing (10) and disengage the resilient tabs (213) of the longitudinal hub (21) from the shoulders (181) on the inner linear surfaces (18) when the actuator (30) is pressed into the casing (10). Each actuating tab (32) has an outer surface (321) and a latch (322). The outer surfaces (321) are slidably mounted respectively against the rear longitudinal surfaces of the inner linear surfaces (18). The latches (322) are wedge shaped, are formed respectively on the outer surfaces (321) of the actuating tabs (32), engage the through holes (141,142) respectively in the top and bottom of the casing (10) to hold the lancet assembly (20) in the casing (10) and disengage from the through holes (14) when the actuator (30) is pressed into the casing (10).

A used needle (211) of the disposable lancet device cannot be used again because the spring (40) is not strong enough to overcome the resistance of the inclined limits (182) against the resilient tabs (213) on the longitudinal hub (21) of the lancet assembly (20). Therefore, a used needle (211) cannot protrude from the casing and accidentally prick anyone.

Even though numerous characteristics and advantages of the present invention have been set forth in the foregoing description, together with details of the structure and features of the invention, the disclosure is illustrative only. Changes may be made in the details, especially in matters of shape, size, and arrangement of parts within the principles of the invention to the full extent indicated by the broad general meaning of the terms in which the appended claims are expressed.

What is claimed is:

1. A disposable lancet device comprising:
a casing being a hollow elongated hexahedron and comprising
a front end having an opening being circular;
an open rear end;
a top having a through hole formed toward the open rear end;
a bottom having a through hole corresponding to the through hole in the top;
two inner side surfaces facing each other; and
two inner linear surfaces facing each other, being between the inner side surfaces, extending from the open rear end to the front end and converging as they approach the front end, and each inner linear surface having
a front longitudinal surface connected to the front end of the casing;
a rear longitudinal surface connected to the open rear end of the casing;
a middle longitudinal surface formed between the front and rear longitudinal surfaces;
a shoulder formed between the rear longitudinal surface and the middle longitudinal surface; and
an inclined limit formed between the middle longitudinal surface and the front longitudinal surface; and
a lancet assembly being a shaft-like assembly, being mounted in the casing through the open rear end and having a longitudinal hub having
a top;
a bottom;
two sides;
a front end;
a rear end;
a needle being mounted in and protruding coaxially from the front end of the longitudinal hub;
a post being formed on and extending coaxially from the rear end of the longitudinal hub; and
two resilient tabs being formed respectively on the top and bottom of the longitudinal hub at the rear end, extending toward the front end of the longitudinal hub and diverging from each other, and each resilient tab having a front edge;
a spring being mounted around the post of the longitudinal hub of the lancet assembly, pressing against the rear end of the longitudinal hub and having
a front end; and
a rear end; and
an actuator mounted slidably in the rear end of the casing and around the rear end of the longitudinal hub to compress the spring and having
a pushbutton assembly being mounted slidably in the open rear end of the casing and having
a cylindrical body being hollow to hold the rear end of the spring and having
an open front end mounted around the spring; and
a closed rear end protruding from the open rear end of the casing; and
two mounting keys formed at and protruding out from the front end of the cylindrical body, slidably mounted between the inner side surfaces of the casing and slidably pressing respectively against the rear longitudinal surfaces of the inner linear surfaces, each mounting key having a front outer edge; and
two actuating tabs being formed at and extending longitudinally respectively from the front outer edges of the mounting keys and slidably mounted respectively against the rear longitudinal surfaces of the inner linear surfaces of the casing, and each actuating tab having
an outer surface slidably mounted against one of the rear longitudinal surfaces of the inner linear surfaces; and
a latch being wedge shaped, formed on the outer surface, engaging a corresponding one of the through holes in the casing to hold the lancet assembly in the casing and disengaging from the through hole when the actuator is pressed into the casing.

2. The disposable lancet device as claimed in claim 1, wherein
the inner side surfaces of the casing further comprise two guide slots formed respectively on the inner side surfaces, facing each other and communicating with the front end; and
the longitudinal hub of the lancet assembly further comprises two ribs longitudinally fonned respectively on the sides of the longitudinal hub and slidably engaging the guide slots on the inner side surfaces.

3. The disposable lancet device as claimed in claim 1, wherein
the casing further comprises two recesses formed in the casing, communicating with the open rear end;
the actuator is mounted slidably in the recesses in the casing; and
the pushbutton assembly further comprises two circumferential stops formed near and protruding out from the closed rear end of the cylindrical body.

4. The disposable lancet device as claimed in claim 1, wherein
the front end of the casing further comprises two vertical notches communicating with and extend radially from the opening opposite to each other; and
the lancet assembly further comprises a needle cap removably attached to and extending from the front end of the longitudinal hub, covering the needle and having
a cylindrical body attached to the front end of the longitudinal hub, covering the needle and having
a front end;
a rear end detachably attached to the front end of the longitudinal hub, covering the needle; and
a longitudinal outer surface;
two longitudinal keys formed on the longitudinal outer surface of the body of the needle cap opposite to each other and aligning with and extending through the vertical notches in the front end of the casing; and
a tab formed on and extending from the front end of the longitudinal body of the needle cap.

5. The disposable lancet device as claimed in claim 3, wherein
the recesses are formed respectively in the inner sides surfaces of the casing; and
the cylindrical body of the pushbutton assembly of the actuator is mounted slidably in the recesses.
